(19) United States
(12) Patent Application Publication
(10) Pub. No.: US 2019/0057190 A1
(43) Pub. Date: Feb. 21, 2019

(54) METHOD AND SYSTEM FOR PROVIDING CONTEXT BASED MEDICAL INSTRUCTIONS TO A PATIENT

(71) Applicant: Wipro Limited, Bangalore (IN)

(72) Inventors: Manjunath Ramachandra Iyer, Bangalore (IN); Sethuraman Ulaganathan, Tiruchirapalli (IN); Ghulam Mohiuddin Khan, Bangalore (IN)

(21) Appl. No.: 15/717,982

(22) Filed: Sep. 28, 2017

(30) Foreign Application Priority Data
Aug. 16, 2017 (IN) 201741028971

(51) Int. Cl. G06F 19/00; G06F 17/30; A61B 5/11; A61B 5/16; A61B 5/00

(52) U.S. Cl. CPC G06F 19/3418; G06F 19/325; G06F 19/322; G06F 17/30424; G06F 17/30554; A61B 5/4842; A61B 5/165; A61B 5/744; A61B 5/486; A61B 5/743; A61B 5/1118

(57) ABSTRACT

A method and system for providing context based medical instructions to a patient is described. The method includes receiving patient data including patient profile and treatment stage associated with the patient. The method further includes determining a physical state of the patient based on continuous monitoring of activities of the patient. The physical state indicates receptive capability of the patient. Further, the method includes generating context based medical instructions based on the physical state and the patient data. The context based medical instructions are delivered to the patient. Further, the method includes monitoring an emotional state of the patient while the patient is performing the context based medical instructions. The method further includes generating dynamically updated context based medical instructions based on the emotional state of the patient. The emotional state indicates patient's interest for receiving the updated context based medical instructions.

### TECHNICAL FIELD

The present subject matter is related, in general to remote monitoring of a patient and more particularly, to a method and system for providing context based medical instructions to the patient.

### BACKGROUND

In a health monitoring environment, one primary objective is to ensure that a patient is timely following medical instructions and taking medicines on time. When the patient is in a medical premise, it is easy to monitor the activities of the patient. The patient is monitored by using medical equipment or during regular checkup sessions. However, with such monitoring, it is not feasible to provide personalized attention to the patient.

In such a scenario, standard medical instructions or alerts are generally sent to the patient which may benefit the patient to some extent. This is because, physical and emotional state of the patient varies from time to time. Further, these factors may also vary from one patient to another patient. Thus, the standard medical instructions do not suit requirement of every patient. It becomes a challenge to monitor activities of the patient in real-time. Further, another challenge faced is configuring and delivering the medical instructions based on the monitored activities of the patient.

### SUMMARY

Disclosed herein is a method of providing context based medical instructions to a patient. The method includes receiving patient data including patient profile and treatment stage associated with the patient. The method further includes determining a physical state of the patient based on continuous monitoring of activities of the patient. The physical state indicates receptive capability of the patient. The method further includes generating one or more context based medical instructions based on the physical state and the patient data. Further, the one or more context based medical instructions are delivered to the patient. The method further includes monitoring an emotional state of the patient while the patient is following the one or more context based medical instructions. The method further includes generating dynamically one or more updated context based medical instructions based on the emotional state of the patient. Further, the emotional state indicates interest of the patient for receiving the one or more updated context based medical instructions.

Further, the present disclosure relates to a health monitoring system for providing context based medical instructions to a patient. The health monitoring system includes a processor and a memory communicatively coupled to the processor. The memory stores processor-executable instructions, which, on execution, causes the processor to receive patient data including patient profile and treatment stage associated with the patient. The processor further determines a physical state of the patient based on continuous monitoring of activities of the patient. The physical state indicates receptive capability of the patient. Further, the processor generates one or more context based medical instructions based on the physical state and the patient data. The one or more context based medical instructions are delivered to the patient. The processor further monitors an emotional state of the patient while the patient is following the one or more context based medical instructions. Further, the processor generates dynamically one or more updated context based medical instructions based on the emotional state of the patient. The emotional state indicates interest of the patient for receiving the one or more updated context based medical instructions.

Furthermore, the present disclosure relates to a non-transitory computer readable medium including instructions stored thereon that when processed by at least one processor cause a health monitoring system to perform the acts of receiving patient data including patient profile and treatment stage associated with the patient. The instructions further cause the at least one processor to determine a physical state of the patient based on continuous monitoring of activities of the patient. The physical state indicates receptive capability of the patient. Further, the instructions further cause the at least one processor to generate one or more context based medical instructions based on the physical state and the patient data. The one or more context based medical instructions are delivered to the patient. The instructions further cause the at least one processor to monitor an emotional state of the patient while the patient is performing the one or more context based medical instructions. The instructions further cause the at least one processor to generate dynamically one or more updated context based medical instructions based on the emotional state of the patient. The emotional state indicates interest of the patient for receiving the one or more updated context based medical instructions.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 is an exemplary environment for providing context based medical instructions to a patient in accordance with some embodiments of the present disclosure;

FIG. 2 is a detailed block diagram illustrating a health monitoring system for providing context based medical instructions to a patient in accordance with some embodiments of the present disclosure;

FIG. 3 is a flowchart illustrating a method of providing context based medical instructions to a patient in accordance with some embodiments of the present disclosure; and

FIG. 4 is a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the spirit and the scope of the disclosure.

The terms “comprises”, “comprising”, “includes”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to a health monitoring system for providing context based medical instructions to a patient. When the patient is admitted in the medical premise, monitoring the activities and health of the patient is feasible. The hospital staff may continuously keep their eyes on the patient's health status during regular visits or checkup sessions or by using various medical equipment. However, such monitoring fails to provide personalized attention to the patients. In other words, it becomes a challenge to check whether the patient is taking his/her medicines on time or sincerely following the medical prescriptions suggested by a medical practitioner.

To overcome the above challenge, the health monitoring system of the present disclosure monitors the activities of the patient when the patient leaves the medical premise or the hospital. The present disclosure focuses on analyzing the real-time behavior or activities of the patient and then delivering relevant content to the patient. Such analysis is important because lifestyle and behavior may vary from one patient to another patient. For example, a patient who is a software professional may maintain a sophisticated lifestyle when compared to another patient who is a farmer or a labor. The health monitoring system considers the above factors (lifestyle and behavior) while generating and delivering the relevant content to the patient.

According to embodiments of present disclosure, the relevant content is the context based medical instructions which includes various content like a video or an audio about exercise or yoga, a text message about medical procedures/routines to be followed by the patient, list of foods and medicines to be taken and the like. Once the context based medical instructions are delivered, the health monitoring system now analyzes interest shown by the patient while watching, listening, or reading the context based medical instructions. This analysis helps the health monitoring system understand psychology of the patient. Based on the understanding, the health monitoring system decides whether to retain/update/replace the previously sent context based medical instructions. This way, the patient gets personalized attention even when the patient is at the remote location. Also, the health monitoring system may learn about the different behaviors of the patient over time which may help in generating more relevant context based medical instructions.

In the following detailed description of the embodiments of the disclosure, reference is made to the accompanying drawings that form a part hereof, and which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

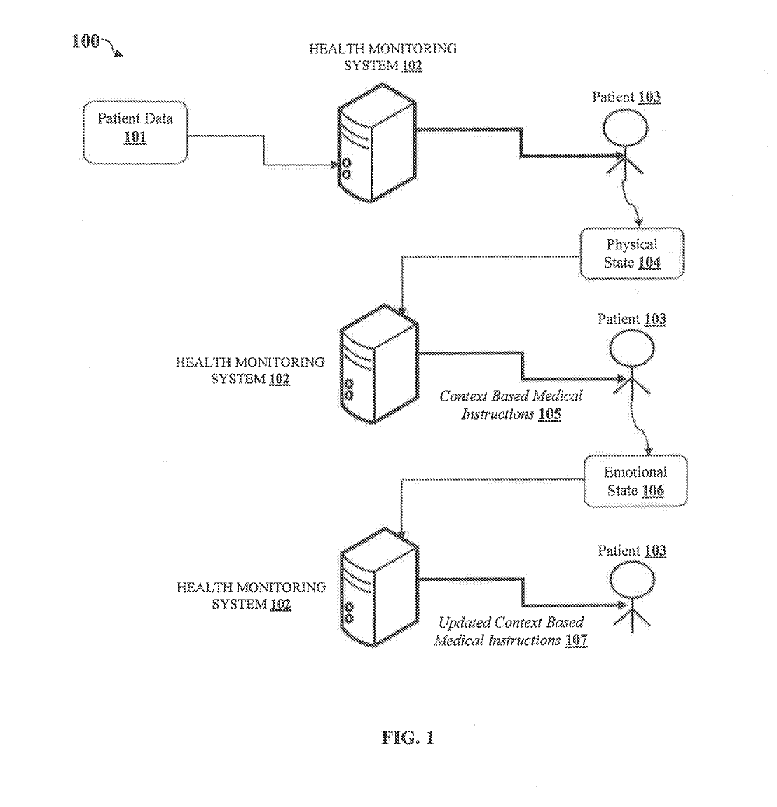

FIG. 1 is an exemplary environment for providing context based medical instructions to a patient in accordance with some embodiments of the present disclosure.

The environment 100 includes patient data 101, health monitoring system 102, patient 103, physical state 104 of the patient 103, context based medical instructions 105, an emotional state 106 of the patient 103, and updated context based medical instructions 107. The patient data 101 may include patient profile and treatment stage associated with the patient 103. The patient data 101 may be collected during the patient's 103 stay in a medical premise, for example a hospital. According to an embodiment, the patient data 101 may be stored in a patient database (not shown in figure) which may either be a part of the health monitoring system 102 or may be external to the health monitoring system 102.

As can be seen from FIG. 1, the working of the health monitoring system 102 has been illustrated in three stages. In a first stage (uppermost part), the health monitoring system 102 may receive the patient data 101 which may include details about the patient 103 and his/her medical information. Apart from receiving the patient data 101, the health monitoring system 102 may continuously monitor activities of the patient 103. For monitoring the activities, according to an embodiment, the health monitoring system 102 may be implemented as an application within a device of the patient 103.

According to this embodiment, the health monitoring system 102 may utilize various components of the device, for example camera, location sensors and the like for sensing the physical activities of the patient 103. Apart from the components, the health monitoring system 102 may also utilize inbuilt sensors for analyzing vital parameters of the patient 103, for example blood glucose, temperature, weight, blood pressure, heart beat and the like. Based on above mentioned physical activities and the vital parameters, the physical state 104 of the patient 103 is determined. The determining of the physical state 104 helps the health monitoring system 102 understand about receptive capability of the patient 103.

Once it is determined that the patient 103 is in a physically fit condition, the health monitoring system 102, based on the patient data 101, generates the context based medical instructions 105 and delivers it to the device (for example, mobile device or smartphone) of the patient 103. The delivery of the context based medical instructions 105 is shown in a second stage of FIG. 1.

Now, the health monitoring system 102 monitors the interest shown by the patient 103 while performing (that is, following, reading, listening, or watching) the context based medical instructions 105. According to an embodiment, the health monitoring system 102 may also track or monitor activities of the patient 103 on internet or on social media to learn about likes and dislikes of the patient 103 on a particular content or topic. This helps the health monitoring system 102 to determine the emotional state 106 of the patient 103.

Based on the emotional state 106, the health monitoring system 102 determines whether to retain the context based medical instructions 105 or to generate updated context based medical instructions 107. The updated context based medical instructions 107 may be a modification of the previously generated context based medical instructions 105 or may be entirely new content. The updated context based medical instructions 107 is now delivered to the patient 103, as shown in a third stage of FIG. 1. This way, not only the physical activities but the interest (likes, dislikes) of patient 103 is taken into account while generating and delivering the medical instructions.

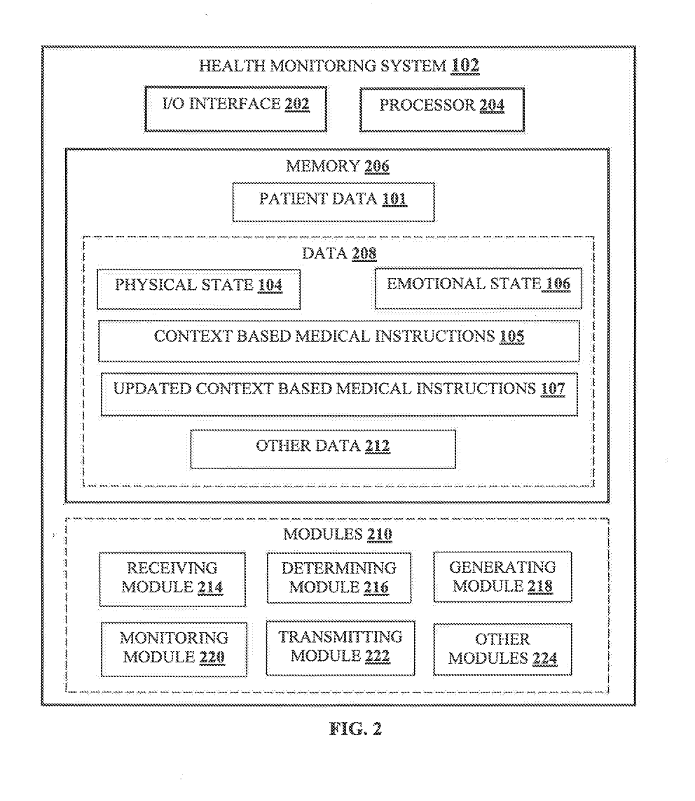

FIG. 2 is a detailed block diagram illustrating a health monitoring system for providing context based medical instructions to a patient in accordance with some embodiments of the present disclosure.

The health monitoring system 102 includes an I/O interface 202, a processor 204, a memory 206, data 208, and modules 210. The memory 206 is communicatively coupled to the processor 204. The processor 204 is configured to perform one or more functions of the health monitoring system 102 for providing context based medical instructions to a patient 103. In one implementation, the memory 206 includes patient data 101 including patient profile and treatment stage associated with the patient 103. The memory 206 further includes data 208. In an embodiment, the data 208 may include, without limitation, the physical state 104, the emotional state 106, the context based medical instructions 105, the updated context based medical instructions 107, and other data 212.

In one embodiment, the data 208 may be stored within the memory 206 in the form of various data structures. Additionally, the aforementioned data 208 can be organized using data models, such as relational or hierarchical data models. The other data 212 may store data, including temporary data and temporary files, generated by the modules 210 for performing the various functions of the health monitoring system 102.

In an embodiment, the physical state 104 may include information pertaining to physical activities and vital parameters of the patient 103. For example, the physical state 104 may include information about consciousness of the patient 103. The physical state 104 helps the health monitoring system 102 understand about the receptive capability of the patient 103. Stated another way, the physical state 104 provides a physical ability of the patient 103 for reading or watching or listening any content/medical instructions.

In an embodiment, the context based medical instructions 105 may include a set of medical instructions in a form of an audio, video, text or combination thereof suitable for the patient 103. The context based medical instructions 105 are generated based on the physical state 104 and the patient data 101. The patient data 101 provides an insight about the patient's medical history and ongoing treatment which helps the health monitoring system 102 determine what kind of medical instructions is suitable for the patient 103. Further, as discussed above, the physical state 104 helps in determining whether the patient 103 is fit or not for receiving the context based medical instructions 105.

In an embodiment, the emotional state 106 may include information pertaining to interest, likes, dislikes of the patient 103 about a topic or content present to him/her. When the context based medical instructions 105 are presented to the patient 103, the health monitoring system 102 monitors the interest level of the patient 103. The patient 103 may like or dislike the context based medical instructions 105.

In an embodiment, the updated context based medical instructions 107 may include a modified version of the context based medical instructions 105 generated previously or may be new set of medical instructions. The updated context based medical instructions 107 may be dynamically generated upon understanding the emotional state 106 of the patient 103.

In some embodiments, the data 208, as discussed above, may be processed by one or more modules 210 of the health monitoring system 102. In one implementation, the one or more modules 210 may be stored as a part of the processor 204. In another implementation, the one or more modules 210 may be communicatively coupled to the processor 204 for performing one or more functions of the health monitoring system 102. The modules 210 may include, without limiting to, a receiving module 214, a determining module 216, a generating module 218, a monitoring module 220, a transmitting module 222, and other modules 224.

As used herein, the term ‘module’ refers to an application specific integrated circuit (ASIC), an electronic circuit, a processor (shared, dedicated, or group) and memory that execute one or more software or firmware programs, a combinational logic circuit, and/or other suitable components that provide the described functionality. In an embodiment, the other modules 224 may be used to perform various miscellaneous functionalities of the health monitoring system 102. It will be appreciated that such modules 210 may be represented as a single module or a combination of different modules.

In an embodiment, the receiving module 214 may receive the patient data 101 including patient profile and treatment stage associated with the patient 103. The patient profile may include personal details like name, age, sex, address, and nationality of the patient 103. Further, the patient profile may also include other information of the patient 103 provided by the caretakers at the time of admission in the medical premise, for example sleep deprivation of the patient 103. Whereas, the treatment stage may include information about disease from which patient 103 is suffering and type of treatments applicable for the patient 103. In one embodiment, the generation of the patient data 101 may be an offline process, in which, a dedicated system placed in the medical premise may generate the patient data 101 when the patient 103 is admitted in the medical premise. However, in another embodiment, the patient data 101 may be generated by the health monitoring system 102. The patient data 101 generated may be stored in a patient database which may be a part of the health monitoring system 102 or may be external to the health monitoring system 102.

According to some embodiments, the patient data 101 is generated by capturing action data and patient specific data when the patient 103 is in the medical premise. Once the patient 103 is admitted, one or more gestures (action data) of the patient may be captured to understand about his/her physical actions. Further, the patient specific data includes medical history of the patient 103 provided by one or more personnels associated with the patient 103.

Once the patient leaves the medical premise after or during the treatment, the challenge is to monitor the patient's 103 activities remotely and delivering specific content to them. The health monitoring system 102 of the present disclosure overcomes this challenge by continuously monitoring the activities of the patient 103. For continuous monitoring, the health monitoring system 102 may utilize various components like camera and inbuilt sensors of a device (mobile device or smartphone) associated with the patient 103. Based on the continuous monitoring, the physical state 104 of the patient 103 is determined by the determining module 216 of the health monitoring system 102.

For example, if an image captured by the camera shows that eyes of the patient 103 is closed, then the determining module 216 may determine that the patient 103 is in a sleeping state and may not able to receive the medical instructions. Similarly, if eyes are open in the captured image, the determining module 216 may determine that the patient 103 is in a conscious state and may be fit for receiving the medical instructions. Not only the images, but videos and voice of the patient 103 may also be captured for determining the physical state 104 of the patient 103.

According to some embodiments, the inbuilt sensors may be used for determining the vital parameters of the patient 103, for example blood glucose, temperature, weight, blood pressure, heart beat and the like. The determination of the vital parameters also helps the determining module 216 in determining the physical state 104 of the patient 103. Thus, the challenge of determining whether the patient's 103 fitness or receptive capability is overcome by determining the physical state 104 of the patient based on the above discussed parameters.

Once the physical state 104 of the patient 103 is determined, the next step is to generate content or medical instructions which may be specific to the patient 103. In an embodiment, the generating module 218 of the health monitoring system 102, may generate the context based medical instructions 105 based on the physical state 104 and the patient data 101 associated with the patient 103. Consideration of the patient data 101, for instance the patient's medical history and type of ongoing treatment, helps the health monitoring system 102 to generate personalized medical instructions for the patient 103. Consideration of the physical state 104 helps the health monitoring system 102 to determine the capability of the patient 103 to accept the medical instructions. It also helps in determining appropriate time for delivering the medical instructions which is explained in subsequent paragraphs of the specification.

Once the context based medical instructions 105 are delivered, the next challenge is to understand the interest level of the patient 103. If the patient 103 shows no or less interest while performing (that is, following, reading, listening, watching) the context based medical instructions 105, then even a specific or personalized content may not be useful for the patient 103. For example, if the context based medical instructions 105 is an audio message of different exercise and the patient 103 is interested in video content, then the patient 103 may not find the audio message useful.

Thus, to overcome this challenge, the monitoring module 220 of the health monitoring system 102 may monitor the emotional state 106 of the patient 103 while the patient 103 is performing (that is, following, reading, listening, watching) the context based medical instructions 105. For monitoring the emotional state 106, according to one embodiment, the monitoring module 220 may analyze facial expressions of the patient 103 while the patient 103 is performing the context based medical instructions 105. According to another embodiment, the monitoring module 220 may also analyze activities of the patient 103 on social media to understand his/her likes and dislikes for determining the emotional state 106.

Once the emotional state 106 (likes, dislikes, interest) is determined, in next step, the generating module 218 may generate dynamically updated context based medical instructions 107 based on the emotional state 106 of the patient 103. Referring back to above example, if the patient 103 is inclined towards the video content, then the context based medical instructions 105 which is delivered in the audio format may be converted into the video format and delivered to the patient 103. However, it may happen that the patient 103 may dislike the content either in the audio or the video format. In this scenario, the generating module 218 may dynamically generate the updated context based medical instructions 107 based on the current interest level of the patient 103.

According to an embodiment, the context based medical instructions 105 and the updated context based medical instructions 107 may include various patient specific content which may be in a video format, an audio format, or text format. For example, the medical instructions (105 and 107) may be a video or audio about an exercise or yoga, a text message about medical procedures or routines to be followed by the patient 103, list of foods and medicines to be taken and the like.

According to an embodiment, the determining module 216 may determine a suitable time for delivering the context based medical instructions 105 and/or the updated context based medical instructions 107 to the patient 103. For example, when the patient 103 is awake or in a position to read, listen or watch, then only the medical instructions are delivered. According to an embodiment, the transmitting module 222 of the health monitoring system 102 transmits the context based medical instructions 105 and/or the updated context based medical instructions 107 at the suitable time to the device associated with at least one of the patient 103 and one or more caretakers associated with the patient 103. The one or more caretakers may be family members or friends or any other person who is taking care of the patient 103.

According to further embodiments of the present disclosure, the health monitoring system 102 may generate an animated character or human-like character for presenting the context based medical instructions 105 and/or the updated context based medical instructions 107 on the device of the patient 103. The animated character may belong to a person associated with a favorite list of the patient. The favorite list may include patient's wife or husband, patient's parents, kids or a friend of the patient 103.

According to further embodiments of the present disclosure, the health monitoring system 102 may determine a time period taken by the patient 103 for performing (following, reading, listening or watching) the context based medical instructions 105 and the updated context based medical instructions 107. The objective of determining the time period is to check that to how much extent the patient 103 is interested in the medical instructions delivered to him/her. It may happen that in the beginning the patient 103 seems to be interested in a particular content, but after some time the patient 103 may get bored and may not like to continue with that particular content.

In such scenario, the health monitoring system 102 may compare the time period with a time threshold indicating a minimum time expected to be taken by the patient 103 for performing the context based medical instructions 105 and the updated context based medical instructions 107. The comparison helps the health monitoring system 102 understand at which moment the patient 103 started disliking the content, or in other scenario, the patient 103 is showing more interest on the delivered content.

Based on the comparison, if it is determined that the time period is less than the time threshold, then it indicates that the patient 103 is having less interest on the delivered content. In this case, the health monitoring system 102 summarizes the context based medical instructions 105 and/or the updated context based medical instructions 107.

In another scenario, if it is determined that the period is greater than the time threshold, then it indicates that patient 103 is showing more or extra interest on the delivered content. Now in this case, the health monitoring system 102 may augment the context based medical instructions 105 and/or the updated context based medical instructions 107.

According to further embodiments of the present disclosure, the health monitoring system 102 may receive one or more queries from at least one of the patient 103 and caretakers. According to an embodiment, the one or more queries may be related to the context based medical instructions 105. For example, the patient 103 may ask “how long should I not take oily food?”. The answer for this particular query may generic or specific. The generic answer may depend upon the patient's 103 health condition and the specific or personalized answer depends upon the food habits, type of job and lifestyle of the patient 103. Thus, based on current health status of the patient 103, the health monitoring system 102 may process the queries to determine responses corresponding to the queries. Once a response is determined, the health monitoring system 102 may display the response on the device associated with the patient 103 and the caretakers.

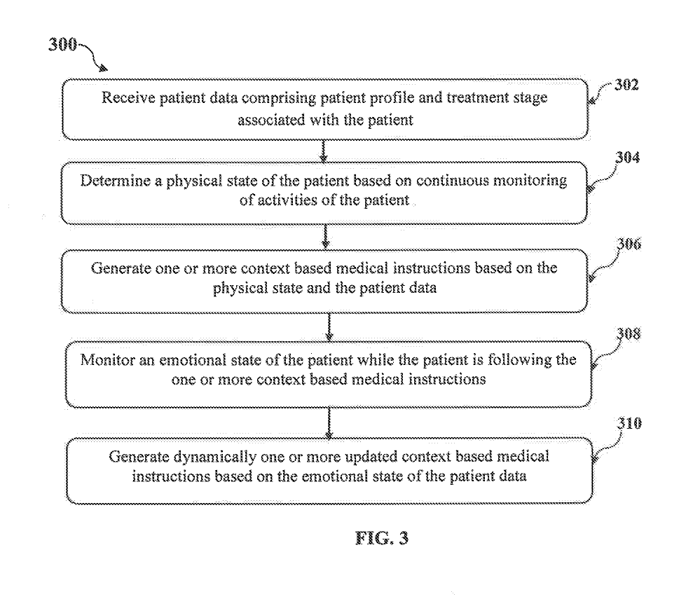

FIG. 3 is a flowchart illustrating a method of providing context based medical instructions to a patient in accordance with some embodiments of the present disclosure.

As illustrated in FIG. 3, the method 300 includes one or more blocks illustrating a method of providing context based medical instructions 105 to a patient 103 using a health monitoring system 102. The method 300 may be described in the general context of computer executable instructions. Generally, computer executable instructions can include routines, programs, objects, components, data structures, procedures, modules, and functions, which perform specific functions or implement specific abstract data types.

The order in which the method 300 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block 302, the method 300 includes receiving, by the health monitoring system 102, patient data 101 including patient profile and treatment stage associated with the patient 103. As explained above with reference to FIG. 2, the patient data 101 is generated by capturing action data and patient specific data when the patient 103 is in a medical premise. The action data includes one or more gestures of the patient 103, whereas the patient specific data includes medical history of the patient provided by one or more personnel associated with the patient 103. Further, the patient data 101 is stored in a patient database accessible by the health monitoring system 102.

At block 304, the method 300 includes determining, by the health monitoring system 102, a physical state 104 of the patient 103 based on continuous monitoring of activities of the patient 103. The physical state 104 indicates receptive capability of the patient 103. As explained above with reference to FIG. 2, the physical state 104 helps the health monitoring system 102 understand whether the patient 103 is fit for receiving any content or not.

At block 306, the method 300 includes generating, by the health monitoring system 102, context based medical instructions 105 based on the physical state 104 and the patient data 101. The generation of the context based medical instructions 105 is explained in the above paragraphs of the specification with reference to FIG. 2. Further, the context based medical instructions 105 are delivered to the patient 103.

At block 308, the method 300 includes monitoring, by the health monitoring system 102, an emotional state 106 of the patient 103 while the patient 103 is performing (that is, following, reading, listening, watching) the context based medical instructions 105.

At block 310, the method 300 includes generating dynamically updated context based medical instructions 107 based on the emotional state 106 of the patient 103. The emotional state 106 indicates interest of the patient for receiving the updated context based medical instructions 107. However, before delivering the context based medical instructions 105 and the updated context based medical instructions 107 to the patient 103, the health monitoring system 102 may determine a suitable time. As explained in the above paragraphs with reference to FIG. 2, the suitable time is a time at which the patient 103 is able to receive the context based medical instructions 105 or the updated context based medical instructions 107 or both. Once the suitable time is determined, health monitoring system 102 transmits the context based medical instructions 105 and the updated context based medical instructions 107 to a device associated with at least one of the patient 103 and one or more caretakers associated with the patient 103.

According to further embodiment of the present disclosure, the method includes providing the context based medical instructions 105 and updated context based medical instructions 107 to the patient through an animated character. As explained in above paragraphs with reference to FIG. 2, the animated character may belong to a person associated with a favorite list of the patient 103. The favorite list may include patient's wife or husband, patient's parents, kids or a friend of the patient 103.

According to further embodiment of the present disclosure, the method includes determining a time period taken by the patient 103 for performing at least one of the context based medical instructions 105 and the updated context based medical instructions 107. As explained in above paragraphs with reference to FIG. 2, the time period shows that to how much extent the patient 103 is interested in the medical instructions delivered to him/her. It may happen that in the beginning the patient 103 seems to be interested in a particular content, but after some time the patient 103 may get bored and may not like to continue with that particular content.

According to further embodiment of the present disclosure, the method includes comparing the time period with a time threshold indicating a minimum time expected to be taken by the patient 103 for performing at least one of the context based medical instructions 105 and the updated context based medical instructions 107. As explained with reference to FIG. 2, the method includes the steps of either summarizing or augmenting the medical instructions (105 and 107) based on the comparison. For example, the method includes the step of summarizing at least one of the context based medical instructions 105 and the updated context based medical instructions 105 when the time period is less than the time threshold. On the other hand, the method includes the step of augmenting at least one of the context based medical instructions 105 and the updated context based medical instructions 105 when the time period is greater than the time threshold.

According explained in above paragraphs with reference to FIG. 2, the method includes receiving one or more queries from at least one of the patient 103 and one or more caretakers of the patient 103. The one or more queries are related to the one or more context based medical instructions 105. The method further includes, processing the one or more queries to determine one or more responses corresponding to the one or more queries. Further, the method includes displaying the one or more responses on a device associated with at least one of the patient and the one or more caretakers.

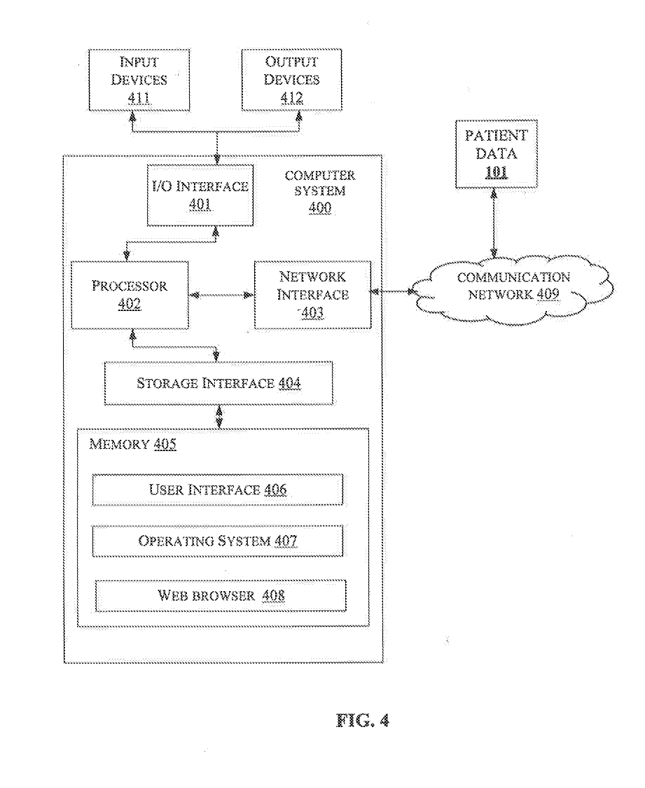

FIG. 4 illustrates a block diagram of an exemplary computer system 400 for implementing embodiments consistent with the present invention. In an embodiment, the computer system 400 can be the health monitoring system 102 which is used for providing context based medical instructions to a patient 103. According to an embodiment, the computer system 400 may receive patient data 101 including patient profile and treatment stage associated with the patient 103. The computer system 400 may include a central processing unit (“CPU” or “processor”) 402. The processor 402 may include at least one data processor for executing program components for executing user- or system-generated business processes. The processor 402 may include specialized processing units such as integrated system (bus) controllers, memory management control units, floating point units, graphics processing units, digital signal processing units, etc.

The processor 402 may be disposed in communication with one or more input/output (I/O) devices (411 and 412) via I/O interface 401. The I/O interface 401 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface 401, the computer system 400 may communicate with one or more I/O devices (411 and 412).

In some embodiments, the processor 402 may be disposed in communication with a communication network 409 via a network interface 403. The network interface 403 may communicate with the communication network 409. The network interface 403 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. The communication network 409 can be implemented as one of the different types of networks, such as intranet or Local Area Network (LAN) and such within the organization. The communication network 409 may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 409 may include a variety of network devices, including routers, bridges, servers, computing devices, storage devices, etc.

In some embodiments, the processor 402 may be disposed in communication with a memory 405 (e.g., RAM 413, ROM 414, etc. as shown in FIG. 4) via a storage interface 404. The storage interface 404 may connect to memory 405 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 405 may store a collection of program or database components, including, without limitation, user/application data 406, an operating system 407, web browser 408 etc. In some embodiments, computer system 400 may store user/application data 406, such as the data, variables, records, etc. as described in this invention. Such databases may be implemented as fault-tolerant, relational, scalable, secure databases such as Oracle or Sybase.

The operating system 407 may facilitate resource management and operation of the computer system 400. Examples of operating systems include, without limitation, Apple Macintosh OS X, UNIX, Unix-like system distributions (e.g., Berkeley Software Distribution (BSD), FreeBSD, Net BSD, Open BSD, etc.), Linux distributions (e.g., Red Hat, Ubuntu, K-Ubuntu, etc.), International Business Machines (IBM) OS/2, Microsoft Windows (XP, Vista/7/8, etc.), Apple iOS, Google Android, Blackberry Operating System (OS), or the like. I/O interface 401 may facilitate display, execution, interaction, manipulation, or operation of program components through textual or graphical facilities. For example, I/O interface may provide computer interaction interface elements on a display system operatively connected to the computer system 400, such as cursors, icons, check boxes, menus, windows, widgets, etc. Graphical User Interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, the computer system 400 may implement a web browser 408 stored program component. The web browser may be a hypertext viewing application, such as Microsoft Internet Explorer, Google Chrome, Mozilla Firefox, Apple Safari, etc. Secure web browsing may be provided using Secure Hypertext Transport Protocol (HTTPS) secure sockets layer (SSL), Transport Layer Security (TLS), etc. Web browsers may utilize facilities such as AJAX, DHTML, Adobe Flash, JavaScript, Java, Application Programming Interfaces (APIs), etc. In some embodiments, the computer system 400 may implement a mail server stored program component. The mail server may be an Internet mail server such as Microsoft Exchange, or the like. The mail server may utilize facilities such as Active Server Pages (ASP), ActiveX, American National Standards Institute (ANSI) C++/C#, Microsoft .NET, CGI scripts, Java, JavaScript, PERL, PHP, Python, WebObjects, etc. The mail server may utilize communication protocols such as Internet Message Access Protocol (IMAP), Messaging Application Programming Interface (MAPI), Microsoft Exchange, Post Office Protocol (POP), Simple Mail Transfer Protocol (SMTP), or the like. In some embodiments, the computer system 400 may implement a mail client stored program component. The mail client may be a mail viewing application, such as Apple Mail, Microsoft Entourage, Microsoft Outlook, Mozilla Thunderbird, and the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### Advantages of the Embodiment of the Present Disclosure are Illustrated Herein

In an embodiment, the present disclosure provides a method of dynamically generating and delivering medical instructions which may be specific to the patient.

In an embodiment, the present disclosure provides a method of updating the medical instructions in a real-time based on interest of the patient or alertness shown by the patient.

In an embodiment, the present disclosure provides effective monitoring of the remote patients.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference Number | Description |
|---|---|
| 100 | ENVIRONMENT |
| 101 | PATIENT DATA |
| 102 | HEALTH MONITORING SYSTEM |
| 103 | PATIENT |
| 104 | PHYSICAL STATE |
| 105 | CONTEXT BASED MEDICAL INSTRUCTIONS |
| 106 | EMOTIONAL STATE |
| 107 | UPDATED CONTEXT BASED MEDICAL |
| | INSTRUCTIONS |
| 202 | I/O INTERFACE |
| 204 | PROCESSOR |
| 206 | MEMORY |
| 208 | DATA |
| 210 | MODULES |
| 212 | OTHER DATA |
| 214 | RECEIVING MODULE |
| 216 | DETERMINING MODULE |
| 218 | GENERATING MODULE |
| 220 | MONITORING MODULE |
| 222 | TRANSMITTING MODULE |
| 224 | OTHER MODULES |

1. A method of providing context based medical instructions to a patient, the method comprising:
receiving, by a health monitoring system, patient data comprising patient profile and treatment stage associated with the patient;
determining, by the health monitoring system, a physical state of the patient based on continuous monitoring of activities of the patient, wherein the physical state indicates receptive capability of the patient;
generating, by the health monitoring system, one or more context based medical instructions based on the physical state and the patient data, wherein the one or more context based medical instructions are delivered to the patient;
monitoring, by the health monitoring system, an emotional state of the patient while the patient is performing the one or more context based medical instructions; and
generating dynamically, by the health monitoring system, one or more updated context based medical instructions based on the emotional state of the patient, wherein the emotional state indicates interest of the patient for receiving the one or more updated context based medical instructions.

2. The method as claimed in claim 1, wherein the patient data is generated by capturing action data and patient specific data when the patient is in a medical premise, and wherein:
the action data comprises one or more gestures of the patient, and
the patient specific data comprises medical history of the patient provided by one or more personnels associated with the patient.

3. The method as claimed in claim 1, wherein the patient data is stored in a patient database accessible by the health monitoring system.

4. The method as claimed in claim 1 further comprising:
determining a suitable time for providing the one or more context based medical instructions and the one or more updated context based medical instructions to the patient.

5. The method as claimed in claim 4 further comprising:
transmitting the one or more context based medical instructions and the one or more updated context based medical instructions at the suitable time to a device associated with at least one of the patient and one or more caretakers associated with the patient.

6. The method as claimed in claim 1 further comprising:
providing the one or more context based medical instructions and the and the one or more updated context based medical instructions to the patient through an animated character.

7. The method as claimed in claim 1 further comprising:
determining a time period taken by the patient for performing at least one of the one or more context based medical instructions and the one or more updated context based medical instructions.

8. The method as claimed in claim 7 further comprising:
comparing the time period with a time threshold indicating a minimum time expected to be taken by the patient for performing at least one of the one or more context based medical instructions and the one or more updated context based medical instructions;
performing, based on the comparing, at least one of:
summarizing at least one of the one or more context based medical instructions and the one or more updated context based medical instructions when the time period is less than the time threshold; and
augmenting at least one of the one or more context based medical instructions and the one or more updated context based medical instructions when the time period is greater than the time threshold.

9. The method as claimed in claim 1 further comprising:
receiving one or more queries from at least one of the patient and one or more caretakers of the patient, wherein the one or more queries are related to the one or more context based medical instructions;
processing the one or more queries to determine one or more responses corresponding to the one or more queries; and
displaying the one or more responses on a device associated with at least one of the patient and the one or more caretakers.

10. The method as claimed in claim 1, wherein the one or more context based medical instructions and the one or more updated context based medical instructions are transmitted to the patient in at least one of a text format, an image format, and a video format.

11. A health monitoring system for providing context based medical instructions to a patient, the health monitoring system comprising:
a processor; and
a memory communicatively coupled to the processor, wherein the memory stores processor-executable instructions, which, on execution, causes the processor to:
receive patient data comprising patient profile and treatment stage associated with the patient;
determine a physical state of the patient based on continuous monitoring of activities of the patient, wherein the physical state indicates receptive capability of the patient;
generate one or more context based medical instructions based on the physical state and the patient data, wherein the one or more context based medical instructions are delivered to the patient;
monitor an emotional state of the patient while the patient is performing the one or more context based medical instructions; and
generate dynamically one or more updated context based medical instructions based on the emotional state of the patient, wherein the emotional state indicates interest of the patient for receiving the one or more updated context based medical instructions.

12. The health monitoring system as claimed in claim 11, wherein the instructions cause the processor to generate the patient data by capturing action data and patient specific data when the patient is in a medical premise, and wherein:
the action data comprises one or more gestures of the patient, and
the patient specific data comprises medical history of the patient provided by one or more personnels associated with the patient.

13. The health monitoring system as claimed in claim 11, wherein the instructions cause the processor to store the patient data in a patient database accessible by the health monitoring system.

14. The health monitoring system as claimed in claim 11, wherein the instructions further cause the processor to determine a suitable time for providing the one or more context based medical instructions and the one or more updated context based medical instructions to the patient.

15. The health monitoring system as claimed in claim 14, wherein the instructions further cause the processor to transmit the one or more context based medical instructions and the one or more updated context based medical instructions at the suitable time to a device associated with at least one of the patient and one or more caretakers associated with the patient.

16. The health monitoring system as claimed in claim 11, wherein the instructions further cause the processor to provide the one or more context based medical instructions and the and the one or more updated context based medical instructions to the patient through an animated character.

17. The health monitoring system as claimed in claim 11, wherein the instructions further cause the processor to determine a time period taken by the patient for performing at least one of the one or more context based medical instructions and the one or more updated context based medical instructions.

18. The health monitoring system as claimed in claim 17, wherein the instructions further cause the processor to:
compare the time period with a time threshold indicating a minimum time expected to be taken by the patient for performing at least one of the one or more context based medical instructions and the one or more updated context based medical instructions;
perform, based on the comparison, at least one of:
summarize at least one of the one or more context based medical instructions and the one or more updated context based medical instructions when the time period is less than the time threshold; and
augment at least one of the one or more context based medical instructions and the one or more updated context based medical instructions when the time period is greater than the time threshold.

19. The health monitoring system as claimed in claim 11, wherein the instructions further cause the processor to:
receive one or more queries from at least one of the patient and one or more caretakers of the patient, wherein the one or more queries are related to the one or more context based medical instructions;
process the one or more queries to determine one or more responses corresponding to the one or more queries; and
display the one or more responses on a device associated with at least one of the patient and the one or more caretakers.

20. The health monitoring system as claimed in claim 11, wherein the instructions cause the processor to transmit the one or more context based medical instructions and the one or more updated context based medical instructions to the patient in at least one of a text format, an image format, and a video format.

21. A non-transitory computer-readable storage medium including instructions stored thereon that when processed by at least one processor cause a health monitoring system to perform operations comprising:
receiving patient data comprising patient profile and treatment stage associated with the patient;
determining a physical state of the patient based on continuous monitoring of activities of the patient, wherein the physical state indicates receptive capability of the patient;
generating one or more context based medical instructions based on the physical state and the patient data, wherein the one or more context based medical instructions are delivered to the patient;
monitoring an emotional state of the patient while the patient is performing the one or more context based medical instructions; and
generating dynamically one or more updated context based medical instructions based on the emotional state of the patient, wherein the emotional state indicates interest of the patient for receiving the one or more updated context based medical instructions.

22. The medium as claimed in claim 21, wherein the patient data is generated by capturing action data and patient specific data when the patient is in a medical premise, and wherein:
the action data comprises one or more gestures of the patient, and
the patient specific data comprises medical history of the patient provided by one or more personnels associated with the patient;

23. The medium as claimed in claim 21, wherein the patient data is stored in a patient database accessible by the health monitoring system.

24. The medium as claimed in claim 21, wherein the instructions further cause the at least one processor to determine a suitable time for providing the one or more context based medical instructions and the one or more updated context based medical instructions to the patient.

25. The medium as claimed in claim 24, wherein the instructions further cause the at least one processor to transmit the one or more context based medical instructions and the one or more updated context based medical instructions at the suitable time to a device associated with at least one of the patient and one or more caretakers associated with the patient.

26. The medium as claimed in claim 21, wherein the instructions further cause the at least one processor to provide the one or more context based medical instructions and the and the one or more updated context based medical instructions to the patient through an animated character.

27. The medium as claimed in claim 21, wherein the instructions further cause the at least one processor to determine a time period taken by the patient for performing at least one of the one or more context based medical instructions and the one or more updated context based medical instructions.

28. The medium as claimed in claim 27, wherein the instructions further cause the at least one processor to:
compare the time period with a time threshold indicating a minimum time expected to be taken by the patient for performing at least one of the one or more context based medical instructions and the one or more updated context based medical instructions;
perform, based on the comparison, at least one of:
summarize at least one of the one or more context based medical instructions and the one or more updated context based medical instructions when the time period is less than the time threshold; and
augment at least one of the one or more context based medical instructions and the one or more updated context based medical instructions when the time period is greater than the time threshold.

29. The medium as claimed in claim 21, wherein the instructions further cause the at least one processor to:
receive one or more queries from at least one of the patient and one or more caretakers of the patient, wherein the one or more queries are related to the one or more context based medical instructions;
process the one or more queries to determine one or more responses corresponding to the one or more queries; and
display the one or more responses on a device associated with at least one of the patient and the one or more caretakers.

30. The medium as claimed in claim 21, wherein the instructions further cause the at least one processor to transmit the one or more context based medical instructions and the one or more updated context based medical instructions to the patient in at least one of a text format, an image format, and a video format.
